(19) United States
(12) Patent Application Publication
(10) Pub. No.: US 2023/0186488 A1
(43) Pub. Date: Jun. 15, 2023

(54) METHODS AND RELATED ASPECTS OF TRACKING MOLECULAR INTERACTIONS

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Guangzhong MA, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/062,261

(22) Filed: Dec. 6, 2022

Related U.S. Application Data
(60) Provisional application No. 63/289,195, filed on Dec. 14, 2021.

(51) Int. Cl. G06T 7/246; G16B 15/00

(52) U.S. Cl. CPC G06T 7/246; G16B 15/00; G06T 2207/10056; G06T 2207/30024

(57) ABSTRACT

Provided herein are methods of tracking molecular dynamics in three dimensions. In some embodiments, the methods include introducing an incident light toward a second surface of a substrate to induce a plasmonic wave at least proximal to a first surface of the substrate. A population of particles is connected to the first surface of the substrate via one or more first biomolecules. In some embodiments, the methods also include detecting a change in position of the particles in the population along at least three dimensions over a duration from a change in intensity of the incident light reflected at an interface of the first surface of the substrate. Related systems and computer readable media are also provided.

### CROSS-REFERENCE TO RELATED APPLICATIONS

This application claims the benefit of U.S. Provisional Application No. 63/289,195 filed Dec. 14, 2021, the disclosure of which is incorporated herein in its entirety.

The application contains a Sequence Listing which has been submitted electronically in .XML format and is hereby incorporated by reference in its entirety. Said .XML copy, created on Dec. 2, 2022, is named “0391.0025.xml” and is 2,954 bytes in size. The sequence listing contained in this .XML file is part of the specification and is hereby incorporated by reference herein in its entirety.

### STATEMENT OF GOVERNMENT SUPPORT

This invention was made with government support under R33 CA235294 and R44 GM126720 awarded by the National Institutes of Health. The government has certain rights in the invention.

### BACKGROUND

Molecules in biological systems perform their function by traveling between different locations and interacting with other molecules. Tracking the motion of single molecule is of fundamental importance to understand molecular heterogeneity, interactions, and myriad intracellular processes. Biomolecules such as protein and DNA are only a few nanometers in size, which requires tracking techniques to have resolution at least comparable to the size to precisely reveal the motion and intramolecular dynamics. On the other hand, the time scale of biomolecular dynamics ranges from microseconds to hours, and thus high temporal resolution and long tracking duration are entailed. Over the past decades, single-molecule fluorescence has emerged as the mainstream technique to track single molecules by incorporating fluorescent dyes into the molecules. However, due to photobleaching and the limited number of photons emitted from a single molecule, the temporal resolution, tracking precision and duration are compromised. To overcome these limitations, nanoparticles are used as an alternative label owing to the strong optical signals. Sub-nanometer precision with microseconds time resolution has been achieved. However, most fluorescence- and particle-based tracking techniques only track the projection of the motion in the imaging plane (x and y directions) for ease of operation, which may lead to biased results due to missing information in the third dimension (axial or z direction). Measuring the third dimension introduces complexity to the existing 2D tracking system, including additional optical components and data processing complexity. One method to determine particle axial movement is to analyze the size and shape of the off-focus patterns arising from diffraction. Other methods utilize high-speed laser scanning in multiple focal planes to localize the particle and move the objective to follow the particle motion via a feedback system. However, it is still challenging to achieve sub-nanometer resolution in z-direction at kHz frame rate. There is a need to develop a simple yet precise 3D tracking technique to measure single-molecule dynamics.

Surface plasmon resonance microscopy (SPRM) is capable of tracking nanoparticles in 3D. Unlike the aforementioned 3D techniques, SPRM extracts axial information directly from the scattering intensity of particles within the evanescent field, which does not introduce additional complexity to the existing system. Since the evanescent field decays exponentially from the surface, the scattering intensity is highly sensitive to z displacement, rendering sub-nanometer resolution in z direction. Together with its nanometer resolution in xy directions and millisecond time resolution, SPRM meets all the requirements for 3D single-molecule dynamics study. Although preliminary studies have demonstrated using SPRM to track single organelle transportation, mechanical oscillation of nanoparticles, and thermal fluctuations of nanoparticles tethered by proteins, its advantage in probing single-molecule dynamics and molecular interactions in 3D still remains to be exploited.

Accordingly, there is a need for effective techniques for tracking molecular dynamics in 3D.

### SUMMARY

This disclosure describes systems and methods for tracking molecular dynamics in at least three dimensions. In some embodiments, for example, SPRM is configured as a multiplexed 3D single-particle tracking technique with sub-nanometer axial resolution at up to kHz frame rate, which allows further analysis of the dynamics of single molecules attached to the particles. In some implementations, short DNA tethered particles are used as a model system to demonstrate the capability of 3D tracking and the benefits in comparison with traditional 2D tracking. In other implementations, the interaction between DNA and a DNA helicase is illustrated to derive the unwinding rate and rotation angle of the helicase from tracking results. In some embodiments, the techniques are used in particle-based immunodetection in terms of identification and removal of non-specific interactions from specific ones. In some embodiments, the camera-based detection comprises the capability of tracking over 100 individual particles simultaneously, which provides enough throughput to generate statistics in a single measurement. The high-precision 3D single particle tracking methods and related aspects disclosed herein provide, for example, new insights into single molecule detection and biosensing.

In one aspect, the present disclosure provides a method of tracking molecular dynamics. The method includes introducing an incident light toward a second surface of a substrate to induce a plasmonic wave at least proximal to a first surface of the substrate, which first surface comprises a population of particles connected to the first surface via one or more first biomolecules. The method also includes detecting a change in position of one or more of the particles in the population along at least three dimensions over a duration, which three dimensions comprise two substantially lateral dimensions and an axial dimension, from a change in intensity of the incident light reflected at an interface of the first surface of the substrate, thereby tracking the molecular dynamics.

In some embodiments, the population comprises between about 2 and about 200 particles. In some embodiments, the methods include detecting changes in position of multiple particles in the population substantially simultaneously. In some embodiments, the methods include detecting changes in position of the particles in the population using a plasmonic imaging technique and/or a microscopic imaging technique. In some embodiments, the methods include detecting changes in position of the particles in the population using surface plasmon resonance microscopy. In some embodiments, the methods further include detecting the change in position of the particles in the population along a rotational dimension.

In some embodiments, the methods include detecting changes in position of the particles in the population with a precision of 10 nanometers (nm) or less (e.g., about 9 nm, about 8 nm, about 7 nm, about 6 nm, about 5 nm, about 4 nm, about 3 nm, about 2 nm, about 1 nm, etc.). In some of these embodiments, the methods include detecting changes in position of the particles in the population in the lateral direction. In some embodiments, the methods include detecting changes in position of the particles in the population with a precision of less than one nanometer in the axial dimension. In some embodiments, the methods include detecting changes in position of the particles in the population at least in the axial dimension at a frame rate of about one kilohertz (kHz) or less.

In some embodiments, the duration comprises a time resolution of 100 milliseconds or less. In some embodiments, the first biomolecules comprise proteins or nucleic acids. In some embodiments, the proteins comprise antibodies. In some embodiments, the methods further include one or more second biomolecules connected to at least some of the particles in the population, wherein the method comprises tracking interactions of the second biomolecules with one or more other biomolecules.

In some embodiments, the second biomolecules comprise proteins (e.g., enzymes, antibodies, nanobodies, etc.), nucleic acids, or other types of biomolecules. In some embodiments, the second biomolecules comprise biocatalysts. In some embodiments, one or more members of the population of particles are connected to the first surface via more than one biomolecule. In some embodiments, the methods include tracking the molecular dynamics in substantially real-time. In some embodiments, the first biomolecules are label-free. In some embodiments, the substrate comprises an Au coating.

In some embodiments, the methods include introducing the incident light via at least one objective lens and/or at least one prism. In some embodiments, the methods include introducing the incident light using a superluminescent diode (SLED).

In some embodiments, the methods include detecting the change in position of the particles in the population along the three dimensions over the duration using a CMOS camera. In some embodiments, the methods include determining an axial position of a given particle using the formula I=I₀e−z/d, where I is the mean image intensity, I₀ is the intensity when the given particle is in contact with the first surface and d is the decay constant of an evanescent field that comprises the given particle. In some embodiments, the methods include detecting specific and/or non-specific interactions of the first biomolecules with one or more other biomolecules.

In another aspect, the present disclosure provides a system for tracking molecular dynamics that includes a substrate having a first surface and a second surface opposite the first surface, wherein the first surface comprises a population of particles connected to the first surface via one or more first biomolecules, and an objective lens and/or a prism disposed proximal to the second surface of the substrate. The system also includes a light source configured to introduce light through the objective lens and/or the prism to induce a plasmonic wave at least proximal to the first surface of the substrate, and a detector configured to collect light reflected from the substrate. In addition, the system also includes a controller that comprises, or is capable of accessing, computer readable media comprising non-transitory computer-executable instructions which, when executed by at least one electronic processor, perform at least: introducing an incident light toward the second surface of the substrate from the light source to induce the plasmonic wave at least proximal to the first surface of the substrate, and detecting a change in position of one or more of the particles in the population along at least three dimensions over a duration, which three dimensions comprise two substantially lateral dimensions and an axial dimension, from a change in intensity of the incident light reflected at an interface of the first surface of the substrate.

In another aspect, the present disclosure provides a computer readable media comprising non-transitory computer executable instruction which, when executed by at least electronic processor, perform at least: introducing an incident light toward a second surface of a substrate from a light source to induce a plasmonic wave at least proximal to a first surface of the substrate, which first surface comprises a population of particles connected to the first surface via one or more first biomolecules, and detecting a change in position of one or more of the particles in the population along at least three dimensions over a duration, which three dimensions comprise at least two substantially lateral dimensions and at least one axial dimension, from a change in intensity of the incident light reflected at an interface of the first surface of the substrate.

In some embodiments of the systems and computer readable media disclosed herein, the population comprises between about 2 and about 200 particles. In some embodiments, an index matching oil is disposed in a gap between the objective lens or the prism and the second surface of the substrate. In some embodiments of the systems and computer readable media disclosed herein, the system comprises a surface plasmon resonance microscopy (SPRM) device. In some embodiments of the systems and computer readable media disclosed herein, the non-transitory computer-executable instructions which, when executed by the electronic processor, further perform at least: detecting the change in position of the particles in the population along a rotational dimension. In some embodiments of the systems and computer readable media disclosed herein, the duration comprises a time resolution of 100 milliseconds or less.

In some embodiments of the systems and computer readable media disclosed herein, the first biomolecules comprise proteins or nucleic acids. In some embodiments of the systems and computer readable media disclosed herein, the proteins comprise antibodies. In some embodiments of the systems and computer readable media disclosed herein, further include one or more second biomolecules connected to at least some of the particles in the population. In some embodiments of the systems and computer readable media disclosed herein, the second biomolecules comprise proteins (e.g., enzymes, antibodies, nanobodies, etc.), nucleic acids, or other types of biomolecules. In some embodiments of the systems and computer readable media disclosed herein, the second biomolecules comprise biocatalysts. In some embodiments of the systems and computer readable media disclosed herein, one or more members of the population of particles are connected to the first surface via more than one biomolecule. In some embodiments of the systems and computer readable media disclosed herein, the first biomolecules are label-free. In some embodiments of the systems and computer readable media disclosed herein, the substrate comprises an Au coating.

### BRIEF DESCRIPTION OF DRAWINGS

FIG. 1 is a flow chart that schematically shows exemplary method steps of tracking molecular dynamics according to some aspects disclosed herein.

FIG. 2 is a schematic diagram of an exemplary system suitable for use with certain aspects disclosed herein.

FIGS. 3A-3H. 3D tracking of particles using surface plasmon resonance microscopy. (a) Experimental setup. Particles are tethered to a gold surface by single-molecule tethers. Incident light is directed to the surface via a microscope objective to excite SPR. The plasmonic images of the particles are collected by the camera in real-time. (b) A SPR image showing 1 μm PS particles tethered by 16 nm dsDNA. The inset is transmitted image of the squared region. (c) Intensity profiles along x and y directions of the SPR pattern of a 1 μm PS particle shown in d (dashed lines). The x profile (top) is fitted with Gaussian distribution and y profile (bottom) is fitted with a right-skewed Gaussian distribution. (d) Localizing the particle by finding the local maxima. The left panel shows an image of a 1 μm PS particle. The center region of the pattern (square) is presented in 3D (right), where the z axis is image intensity. The local maximum is found (dot) and then used for particle localization by TrackMate. (e) Localization precision of SPR tracking. Precision in xy and z directions are determined to be ˜2 nm and 0.44 nm respectively by tracking the relative position between two immobilized particles. The standard deviation (Std) of relative position fluctuation is defined as localization precision. (f) The motion of a streptavidin coated 1 μm PS particle near the surface revealed by SPR 3D tracking. The gold film surface was passivated with MT(PEG)4 to reduce non-specific absorption. The particle showed a c-shaped pattern caused by interaction with the surface followed by random patterns due to Brownian motion. The shadow on xy plane is projection of the 3D pattern. The particle motion was tracked for 8.1 s at 100 fps. (g) Simultaneous 2D tracking via transmitted light. The xy projection of SPR 3D tracking (top panel) and transmitted light tracking (bottom panel) of the same particle show similar patterns. (h) Evaluation of tracking accuracy by comparing the 2D patterns. The x (top panel) or y (bottom panel) coordinates from SPR tracking and transmitted tracking are plotted in the same graph, and both have a correlation factor R²>0.997. The fitted slope (line) in x and y are 0.908 and 1.17, respectively.

FIGS. 4A-4K. The motion of particle tethered by different number of DNA molecules. (a) The motion of a single DNA tethered particle showing a dome pattern in space. The xy coordinates represent the centroid of the particle, and z coordinate represents the distance from the bottom of the particle to the surface. For clarity, the pattern is rotated 90° and 180° in the right panels. (b) Projection of the 3D pattern onto xy plane. (c) Projection of the 3D pattern onto z-axis. (d) The motion of multiple DNA tethered particle shows a section of dome due to the restriction from additional tethers.

The right panels show 90° and 180° rotation of the pattern. (e) Projection of the pattern onto xy plane. (f) Projection of the pattern onto z-axis. (g) The motion of many DNA tethered particles shows that the particle is confined within a small region. The right panels show 90° and 180° rotation of the pattern. (h) and (i) show the projection of the pattern onto xy plane and z-axis, respectively. The tracking frame rate for a, d, and g is 100 fps. (j) Schematic showing a particle with radius of a tethered by a DNA with length of L. The dome (solid line and shadow), which is the largest area that the tethered particle can explore, is a section of sphere with radius of a+L. (k) Distribution of restriction factor R obtained from 121 tethered particles. The tether number decreases from many tethers to a single tether as R increases from 0 to 1.

FIGS. 5A-5J. Tracking the interaction between RecBCD and dsDNA. (a) dsDNA is anchored on the gold film, and RecBCD is modified on the surface of a 100 nm gold nanoparticle (AuNP), which unwinds the dsDNA in the presence of ATP. The remaining length of double strand (L) and the rotation angle of the RecBCD or AuNP (θ) are obtained by tracking the 3D coordinates of the AuNP. (b) Tracking the motion of a RecBCD coated AuNP near the surface. The trajectory of the particle shows Brownian motion and interaction with the surface. (c) Zoom-in of the interaction event shows the motion of AuNP was confined within a nanometer-scaled domain with decreasing L and rotating θ, which indicates the RecBCD was unwinding the DNA. (d) The change of L is obtained from the 3D coordinates in c, where the dots and black curve are raw data and 20 points-smoothed data, respectively. The DNA unwinding rate was determined to be 1.9 bps/s by linear fitting of the curve. (e) Polar graph showing the rotation of RecBCD upon unwinding. The dashed line marks a possible route of rotation. (f) Unwinding rate of 4 individual DNA molecules. For clarity, the beginnings of the curves are aligned at 16 nm. A total of N=14 DNA molecules were measured, the mean rate was 6.8 bps/s with a standard deviation of 6.5 bps/s. (g) L and θ (converted to turns) obtained from 5 DNA molecules. The relation between L and θ were determined by linear fitting of the data, which was 3.7 nm/turn. (h) Control experiment without DNA. The RecBCD coated AuNP bound to the surface via non-specific interaction which showed random fluctuations. (i) L change in non-specific interaction, which was calculated using the coordinates in h. The dots and curve are raw data and 20-point smoothed data respectively. (j) Polar graph showing 0 change in non-specific interaction.

FIGS. 6A-6N. Particle motion reveals the specific binding and non-specific binding of troponin T. (a) Specific binding of troponin T (TnT) in a sandwich immunoassay. TnT is captured by the capture antibody (Cap Ab) immobilized on the surface. The detection antibody (Det Ab) binds to the captured TnT with the Fab domain and captures the 1 μm streptavidin (Strep) coated PS particle via the biotin moiety on the Fc domain. Note that the schematic is not drawn to scale. (b) 3D motion pattern of a representative particle tethered by the antibody-TnT-antibody complex. (c) Non-specific binding in the absence of TnT. (d) 3D pattern of a non-specifically bound particle showing restricted motion. (e) Counts of particles bound to the surface at different TnT concentrations. The 0 ng/L sample was measured in 10 times diluted PBS, and the other samples were measured in serum. (f) Dose-response curve obtained by counting particles that are specifically bound to the surface. The solid curve is linear fitting of the data, and the dashed line marks the detection limit (0.486 ng/L). The error bars in (e) and (f) represent standard deviation determined from 3 imaging regions on the same sensor surface. The particles used in (e) and (f) were 150 nm streptavidin coated gold nanoparticles. (g) The specific binding is flexible and can be modulated by a laminar flow. (h) The motion pattern of a specifically bound particle under four different flow rates or forces. (i) Projection of the pattern in h on xy plane and z axis. (j) The non-specific bond is less flexible and cannot be stretched by the flow. (k) Motion pattern of the non-specifically bound particle in flow. (l) Projection of the pattern in k on xy plane and z axis. (m) Further increasing the flow rate ruptures the tether. Tethers with specific interactions are more difficult to break than those with non-specific interactions. (n) Non-specifically bound particles are almost removed at 10 pN, whereas over 50% specifically bound particles remain on the surface at 30 pN. The particles used here were 150 nm gold nanoparticles and the surface was glass.

FIG. 7 schematically shows aspects of tracking particle motion, including in a rotational dimension, according to some aspects disclosed herein.

### Definitions

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Biomolecule: As used herein, “biomolecule” refers to an organic molecule produced by a living organism. Exemplary biomolecules, include without limitation macromolecules, such as nucleic acids, proteins, peptides, oligomers, carbohydrates, and lipids.

Nucleic Acid: As used herein, “nucleic acid” refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., bromodeoxyuridine (BrdU)), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA, cfDNA, ctDNA, or any combination thereof.

Protein: As used herein, “protein” or “polypeptide” refers to a polymer of at least two amino acids attached to one another by a peptide bond. Examples of proteins include enzymes, hormones, antibodies, and fragments thereof.

Refractive Index: As used herein, the term “refractive index” refers to a ratio of the speed of light in one medium (e.g., air, glass, or a vacuum) to that in another medium. In some embodiments, a refractive index of a given substrate (e.g., an optically transparent glass substrate) exceeds a refractive index of a liquid comprising a ligand being assessed.

Resonance Angle: As used herein, the term “resonance angle” in the context of optically analyzing molecular interactions on substrates refers to an angle of incident light at which resonance occurs. In some embodiments, molecular interactions are assessed by detecting changes or shifts in resonance angles.

### DETAILED DESCRIPTION

Three-dimensional (3D) tracking of surface-tethered single-particle reveals the dynamics of the molecular tether. However, most 3D tracking techniques lack precision, especially in an axial direction, for measuring the dynamics of biomolecules with spatial scale of several nanometers. In some embodiments, the present disclosure provides a plasmonic imaging technique that can track the motion of ˜100 or more tethered particles in 3D simultaneously with sub-nanometer axial precision and single-digit nanometer lateral precision at millisecond time resolution. By tracking the 3D coordinates of tethered particles with high spatial resolution, the techniques of the present disclosure can be used to determine the dynamics of single short DNA and study its interaction with enzymes. In some embodiments, particle motion pattern can be used to identify specific and non-specific interactions in immunoassays. Among other exemplary aspects, the 3D tracking techniques disclosed herein can contribute to the understanding of molecular dynamics and interactions at the single-molecule level.

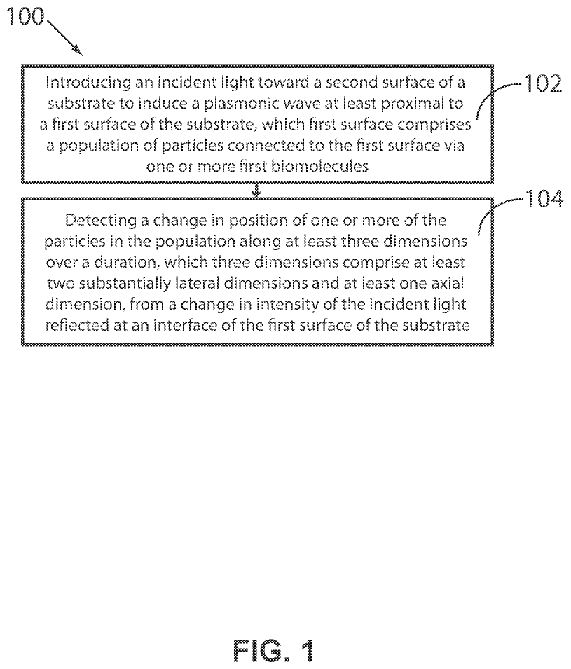

To illustrate, FIG. 1 is a flow chart that schematically shows exemplary method steps of tracking molecular dynamics according to some aspects disclosed herein. As shown, method 100 includes introducing an incident light toward a second surface of a substrate to induce a plasmonic wave at least proximal to a first surface of the substrate, which first surface comprises a population of particles connected to the first surface via one or more first biomolecules (step 102). Method 100 also includes detecting a change in position of one or more of the particles in the population along at least three dimensions over a duration, which three dimensions comprise at least two substantially lateral dimensions and at least one axial dimension, from a change in intensity of the incident light reflected at an interface of the first surface of the substrate, thereby tracking the molecular dynamics (step 104).

In some embodiments, the population comprises between about 2 and about 200 particles. In some embodiments, the methods include detecting changes in position of multiple particles in the population substantially simultaneously. In some embodiments, the methods include detecting changes in position of the particles in the population using a plasmonic imaging technique and/or a microscopic imaging technique. In some embodiments, the methods include detecting changes in position of the particles in the population using surface plasmon resonance microscopy. In some embodiments, the methods further include detecting the change in position of the particles in the population along a rotational dimension.

In some embodiments, the methods include detecting changes in position of the particles in the population with a precision of about 10 nanometers (nm) or less (e.g., about 9 nm, about 8 nm, about 7 nm, about 6 nm, about 5 nm, about 4 nm, about 3 nm, about 2 nm, about 1 nm, etc.). In some of these embodiments, the methods include detecting changes in position of the particles in the population in the lateral direction. In some embodiments, the methods include detecting changes in position of the particles in the population with a precision of less than one nanometer in the axial dimension. In some embodiments, the methods include detecting changes in position of the particles in the population at least in the axial dimension at a frame rate of about one kilohertz (kHz) or less.

In some embodiments, the duration comprises a time resolution of 100 milliseconds or less. In some embodiments, the first biomolecules comprise proteins or nucleic acids. In some embodiments, the proteins comprise antibodies. In some embodiments, the methods further include one or more second biomolecules connected to at least some of the particles in the population, wherein the method comprises tracking interactions of the second biomolecules with one or more other biomolecules. In some embodiments, the second biomolecules comprise proteins (e.g., enzymes, antibodies, nanobodies, etc.), nucleic acids, or other types of biomolecules. In some embodiments, the second biomolecules comprise biocatalysts. In some embodiments, one or more members of the population of particles are connected to the first surface via more than one biomolecule. In some embodiments, the methods include tracking the molecular dynamics in substantially real-time. In some embodiments, the first biomolecules are label-free. In some embodiments, the substrate comprises an Au coating.

In some embodiments, the methods include introducing the incident light via at least one objective lens and/or at least one prism. In some embodiments, the methods include introducing the incident light using a superluminescent diode (SLED). In some embodiments, the methods include detecting the change in position of the particles in the population along the three dimensions over the duration using a CMOS camera. In some embodiments, the methods include determining an axial position of a given particle using the formula I=I₀e−z/d, where I is the mean image intensity, I₀ is the intensity when the given particle is in contact with the first surface and d is the decay constant of an evanescent field that comprises the given particle. In some embodiments, the methods include detecting specific and/or non-specific interactions of the first biomolecules with one or more other biomolecules.

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving molecular interaction data sets or results, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for tracking molecular dynamics as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to molecular interaction data, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: introducing an incident light toward a second surface of a substrate from a light source to induce a plasmonic wave at least proximal to a first surface of the substrate, which first surface comprises a population of particles connected to the first surface via one or more first biomolecules, and detecting a change in position of one or more of the particles in the population along at least three dimensions over a duration, which three dimensions comprise at least two substantially lateral dimensions and at least one axial dimension, from a change in intensity of the incident light reflected at an interface of the first surface of the substrate.

Typically, tracking molecular dynamics data is obtained using device 218. As shown, device 218 includes substrate 224 (e.g., gold coated coverglass) having first surface 232 and second surface 234 opposite first surface 232. First surface 232 comprises a population of particles 220 connected to first surface 232 via first biomolecules 222 (e.g., a nucleic acid, a protein, or the like), which functions like a molecular tether. Directional arrow 221 schematically illustrates particle motion.

Objective 226 is coupled to second surface 234 of substrate 224. Device 218 also includes light source 227 (e.g., a superluminescent diode (SLED)) configured to introduce light (e.g., collimated light) into objective 226 to induce a plasmonic wave at least proximal to first surface 232 of substrate 224. Evanescent field 229 is schematically depicted. In addition, device 218 also includes detector 228 (e.g., a CMOS camera) configured to collect light reflected from an interface between first surface 232 of substrate 224 and first biomolecules 222 and particles 220.

### Example: Three-Dimensional Tracking of Tethered Particles for Probing Nanometer-Scale Single-Molecule Dynamics Using Plasmonic Microscope

Results

Detection Principle

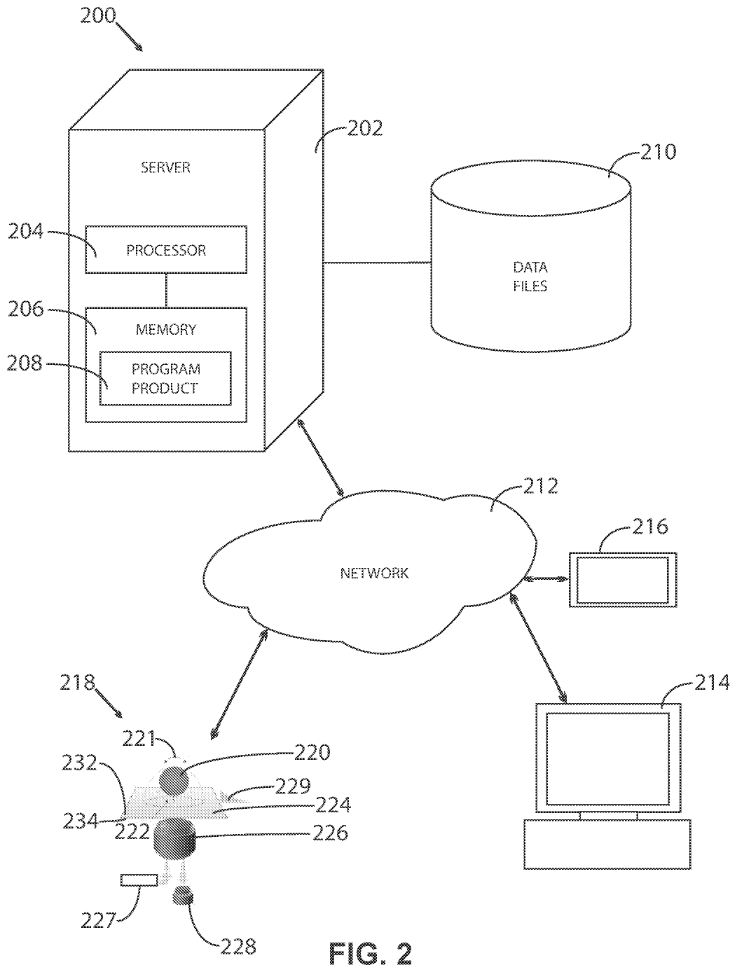

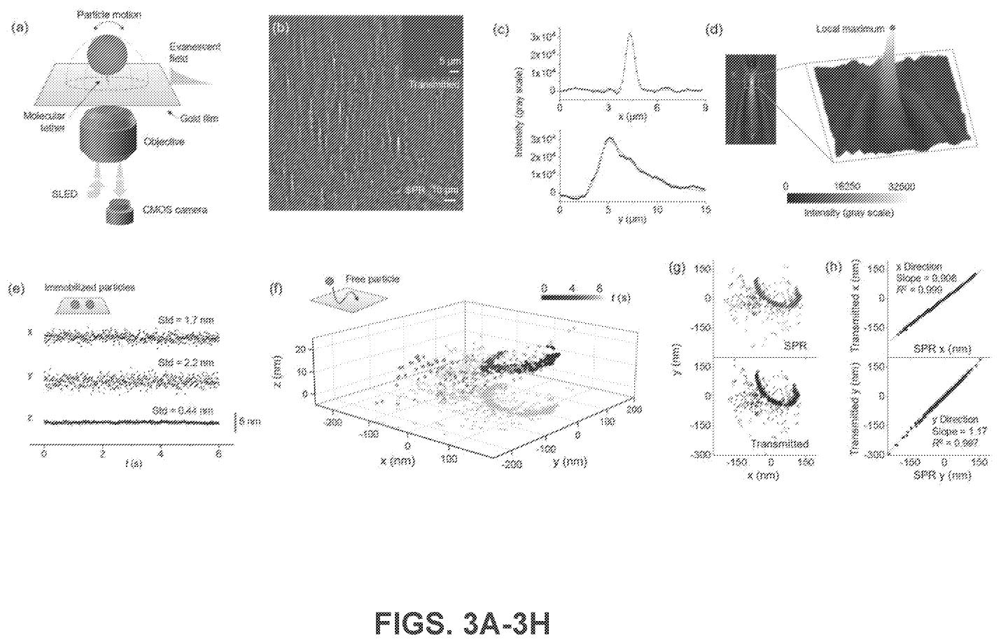

Particles are tethered to a gold surface using DNA or protein molecules. An objective-based plasmonic imaging setup is used for tracking the tethered particles (FIG. 3a). The surface plasmonic wave is excited on the gold surface using a superluminescent diode (SLED), which is then scattered by particles and generates a bright spot with parabolic tails due to interference (FIG. 3b). This pattern is known as the point spread function (PSF) of particle under SPRM. FIG. 3b shows the SPR image of over 100 DNA tethered particles that can be tracked simultaneously. The lateral (xy) intensity profile of each single particle shows a Gaussian distribution in x direction and a skewed Gaussian distribution in y direction, respectively (FIGS. 3c-d). Therefore, the peak position can be used to localize the particle in xy plane. We localize the particles using a single-particle tracking software, TrackMate, which automatically finds the local maximum for each particle and tracks the motion over time. After obtaining the local maximum, the mean intensity of all pixels surrounding the local maximum within a circle of ˜1.5 μm diameter is used to calculate the axial position z, given by I=I₀e−z/d where I is the mean image intensity, I₀ is the intensity when the particle is closely attached to the surface (at z=0) and d is the decay constant of evanescent field (˜100 nm). To evaluate tracking precision, we recorded the relative movement between two particles stuck on the surface at 100 frames per second (fps) for 6 s and calculated the standard deviation in x, y and z directions. The standard deviation is defined as localization precision, which is ˜2 nm in xy, and 0.44 nm in z (FIG. 3e). Due to the exponential decay of evanescent field, localization in z is more precise than xy, which is a unique feature of SPR. We also note that by increasing the incident light intensity, higher localization precision (1 nm in xy and 0.1 nm in z) and temporal resolution (1000 fps) can be achieved (see Discussion). Using the experimental condition and spaciotemporal resolution in FIG. 3e, we tracked the motion of a free 1 μm polystyrene (PS) particle near the surface in 3D as an example. The high resolution revealed detailed nanometer-scale information, including particle-surface interaction (the c-shape pattern) and Brownian motion (the scattered pattern), as shown in FIG. 3f. To demonstrate the tracking accuracy, we introduced a second imaging channel additional to the existing SPR channel, which used transmitted light to simultaneously track the particle motion in 2D. The xy projection of the 3D SPR pattern in FIG. 3f was similar to the pattern obtained by transmitted light tracking (FIG. 3g), except for minor deviation.

The deviation in x and y directions were quantified respectively by constructing correlation curves using the x and y coordinates determined from the two tracking methods (FIG. 3h). The correlation in both directions were strong with R>0.997, indicating SPR has excellent tracking accuracy. However, the slope of the correlation curves in x and y were 0.908 and 1.17, or −9.2% and 17% deviation, respectively. This deviation is linear because the R² is close to 1. Further analysis suggests that the deviation is likely due to fitting algorithm and different imaging principle between SPR and transmitted imaging (see Discussion).

3D Tracking of DNA Tethered Particles

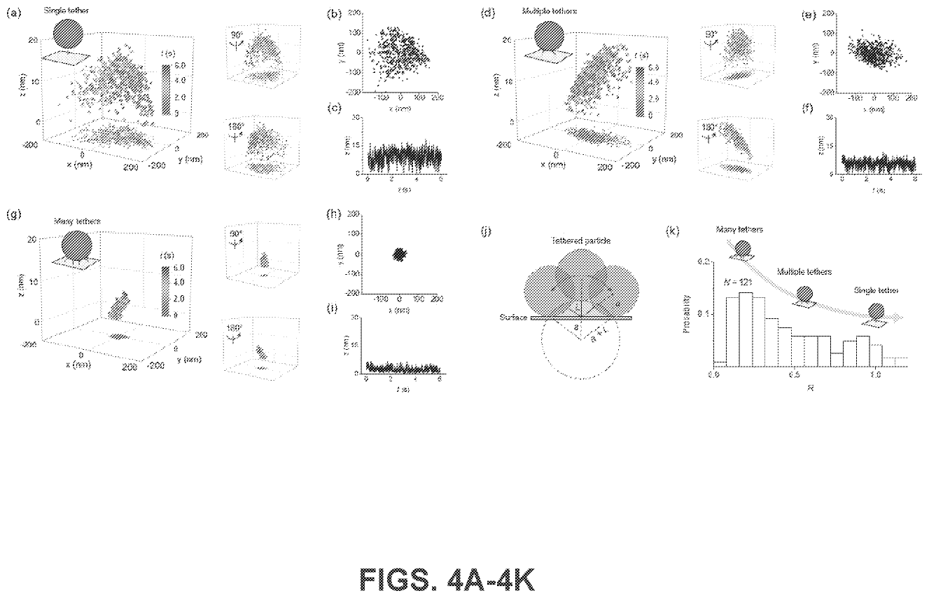

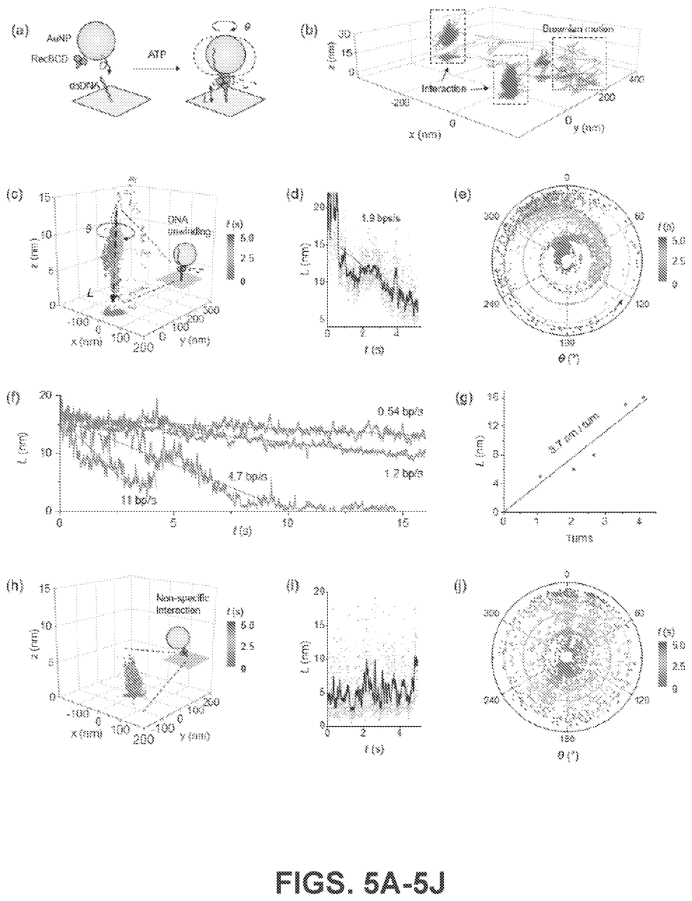

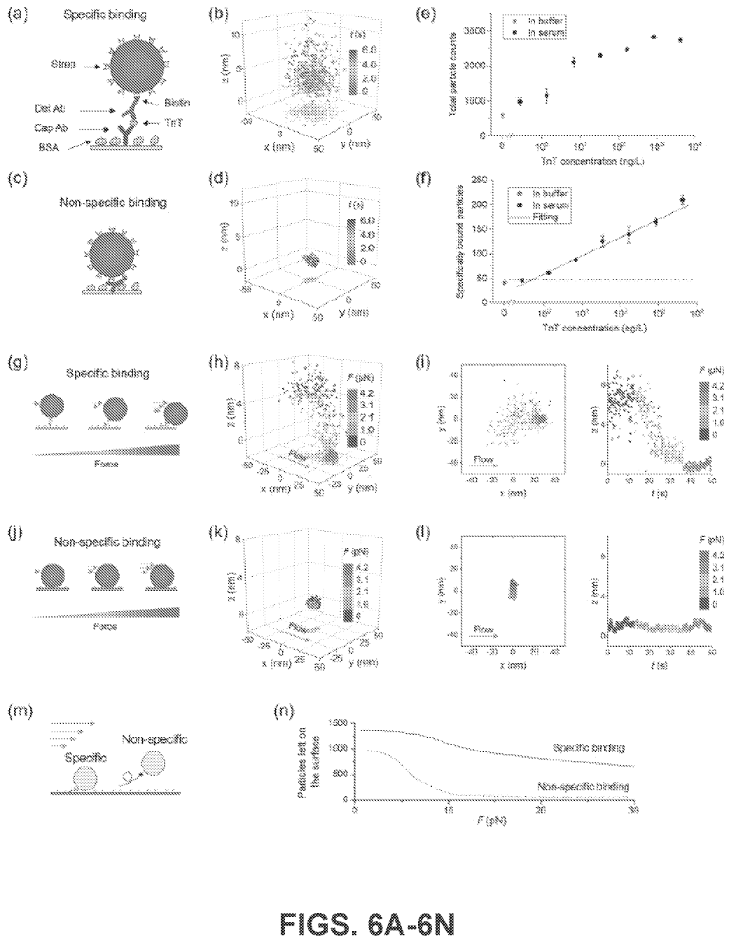

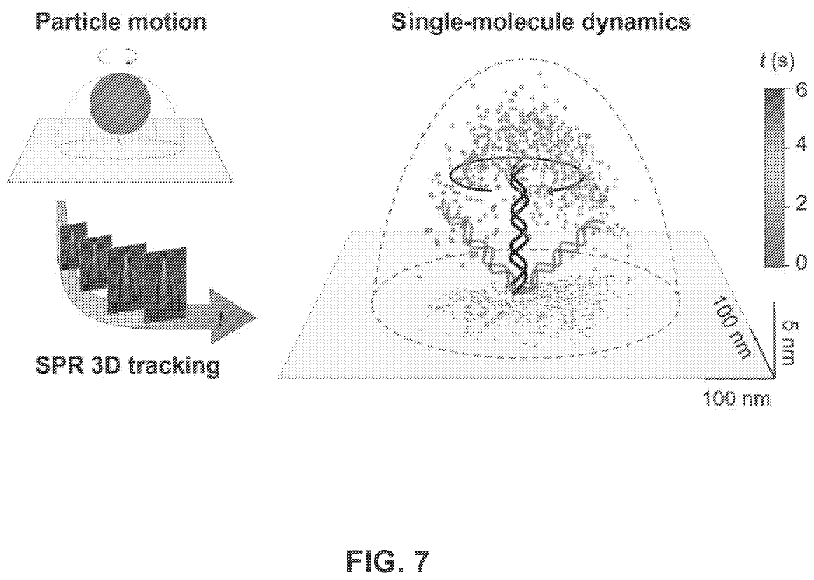

To study the dynamics of nanometer-scaled biomolecules, we tethered particles to the gold surface using short DNA molecules and tracked the particle motion in 3D. The 48 bp (16 nm) double-stranded DNA (dsDNA) was functionalized with a thiol group on one end to couple the gold surface, and a biotin on the other end to capture the 1 μm streptavidin coated PS particle. The density of DNA tether on the surface was adjusted by diluting with spacer molecules to ensure that most particles were tethered by one or a few DNA molecules. In a previous study using bright field 2D tracking, the motion pattern of short rigid dsDNA-tethered particle was correlated with the number of DNA tethering the particle. They found that single, multiple (likely 2 or 3), and many (>3) DNA tethered particles displayed characteristic circular, triangular/stripe, and spot patterns. Intuitively, the 2D patterns observed should be the projection of 3D motion onto the image plane. To test our hypothesis, we tracked 121 tethered particles within a 70 μm×70 μm region at 100 fps for 6 seconds and recorded the motion patterns for each particle in 3D. As expected, the 2D projection showed the same three types of patterns due to single, multiple, and many tethers. In addition, introducing the third dimension reveals more information. For example, particle with one tether has a dome-shaped pattern in space (FIGS. 4a-c), which is due to the free rotation of DNA, while particle with multiple tethers shows a section of the dome (FIGS. 4d-f), because the motion is restricted by the additional tether. Particles tethered by many DNA molecules are confined within a much smaller region, which is also a section of the dome due to extra restriction by excess tethers (FIGS. 4g-i).

The area of particle excursion (A) reflects the restriction exerted on particle by tethers. The largest excursion area (A₁) is attained when there is only one tether, which has a spherical cap or dome shape with a radius determined by both particle size and tether length (FIG. 4j). Thus, the ratio R=A/A₁, is a measure of motion restriction with a maximum of ˜1 for a single tether and approaches to 0 for many tethers. We calculated the excursion area for all the 121 particles mentioned above and constructed a histogram in FIG. 4k. It can be seen that although the DNA tethering was categorized into 3 states (single, multiple and many), the transition between these states in the histogram is very smooth without obvious peaks, implying that one could not accurately quantify the number of tethers merely from the excursion area. We believe that further combining simulation and experimental data with higher spatial resolution and using additional parameters such as spatial probability density for comparison may help to solve this problem in the future. Nevertheless, the 3 states criteria can still serve as an acceptable estimation.

Measuring RecBCD-DNA Interaction

The sub-nanometer tracking resolution allows us to probe the intramolecular interaction dynamics of single molecules. To demonstrate this capability, we tracked the interaction between DNA and a DNA enzyme called RecBCD. RecBCD is a hetero-trimeric complex of helicase and nuclease found in E. coli, which is responsible for initiating the repair of dsDNA breaks in the homologous recombination pathway. When RecBCD binds DNA in the presence of ATP, the two helicase subunits unwind the double stranded DNA from one end to the other. Recent single-molecule studies have shown that this unwinding process is accompanied by the rotation of RecBCD due to the double helix structure of DNA, however, how the rotation is related to the progression of RecBCD on DNA is unclear. This is because by far it is difficult to measure the spatial position and rotation of RecBCD on a single platform simultaneously. DNA length is often measured by magnetic and optical tweezers, while RecBCD rotation is measured by a DNA origami-based method called ORBIT, which is known to persons having ordinary skill in the art. Since our tracking technique can probe the 3D coordinates of RecBCD in space, it is possible to obtain DNA length (L) and RecBCD rotation angle (θ) from the coordinates (FIG. 5a).

To facilitate the tracking, we immobilized RecBCD on 100 nm gold nanoparticles (AuNPs). Immobilization of RecBCD on surface has negligible effects on enzymatic activity if the RecBCD is properly oriented. We functionalized the gold surface of the sensor chip with 48 bp dsDNA and filled the sample cell with 1× NEBuffer and 5 μM ATP, which provided a suitable environment for RecBCD to function. This ATP concentration should initiate the unwinding process at a rate of ˜10 bp/s, which can be readily recorded by the camera at 400 fps. After adding the RecBCD coated AuNPs to the cell, we observed that most AuNPs underwent Brown motion interspersed with transient interactions with the surface, and eventually stuck on the surface. FIG. 5b shows the motion of an AuNP within 26.5 s and two interactions events were found. The spatial dimension of each interaction was ˜50 nm in xy and ˜20 nm in z, indicating the AuNP was trapped within a small region. To find RecBCD-DNA interactions, we zoomed in the interaction patterns and plotted the position of the particle against time. For AuNPs with active RecBCD that unwinds the DNA, we found that the AuNP moved unidirectionally from the distal end of DNA to the anchored end on the surface (FIG. 5c). The 3D coordinates of the AuNP at different timepoint allowed us to extract the position of RecBCD in contact with the DNA and hence calculate the DNA length change. The length decreased from 14 nm to 6 nm in 4 s after the RecBCD coated AuNP binding to the DNA (FIG. 5d). By fitting the length change with a linear model, the reaction rate of RecBCD was determined to be 1.9 nm/s or 6.3 bps/s. Knowing the 3D coordinates also allowed us to extract the rotation of RecBCD around DNA. FIG. 5e shows the angular motion of RecBCD during the unwinding process obtained from the coordinates in FIG. 5c. The spiral pattern in the polar graph suggests the motion is rotation, and the total rotation angle is 960° (˜2.7 turns) within 4 s. The conversion between DNA length and turns was thus determined to be 3.0 nm/turn or 10 bps/turn, consistent with literature value. Additional measurements with several different individual DNA molecules showed that the unwinding rates differed (FIG. 5f) but the relation between length and turn was similar (FIG. 5g).

To confirm the rotation is due to specific interaction between DNA and RecBCD, we performed a control experiment using a surface without DNA (only with spacer molecules). The RecBCD coated AuNPs diffused to the surface and fluctuated within a small region after hitting the surface (FIG. 5h). In contrast to the specific interaction, the non-specific interaction showed a smaller motion range with a constantly fluctuating RecBCD-surface distance of ˜5 nm (FIG. 5i), which was likely due to the fluctuation of RecBCD and spacer molecules on the surface. Moreover, the rotation angle of the AuNP was random rather than spiral (FIG. 5j). An additional control experiment using DNA coated surface but without ATP showed similar results. All these findings imply that the DNA length change and RecBCD rotation that we observed were due to DNA unwinding. We have studied 135 AuNP-surface interactions from 100 AuNPs, but only 16 unwinding events were found. Such low reaction rate (12%) may arise from the unfavorable orientation of RecBCD immobilized on the AuNPs.

Identification of Specific and Non-Specific Interactions in Immunoassay

Besides the DNA, in a more general sense, any molecules or complexes connecting the particle and the surface can act as a tether, and the dynamics of the tether can be probed by tracking the particle. Here we show an example of tracking the particles used in digital ELISA and determine the binding specificity from the motion patterns. Digital ELISA is a recently developed biosensing technique that involves particles as the label for single molecules, e.g. antibodies. We studied the binding of troponin T (TnT), a biomarker for heart diseases, to its antibody using a sandwich immunoassay provided by a commercial ELISA kit. First, the capture antibody was immobilized on the gold surface via NHS/EDC chemistry. The antibody coverage was carefully controlled to avoid multiple tethers binding to the same particle. Then the surface was blocked by 0.1% bovine serum albumin (BSA) to minimize non-specific interactions. Next, 4200 ng/L TnT was introduced to the system and incubated for 30 min to allow binding to the capture antibody. A second TnT antibody, known as the detection antibody, with a biotin moiety in the Fc domain was used to sandwich the captured TnT. The antibody-antigen-antibody complex was tagged with 1 μm streptavidin coated PS particle via streptavidin-biotin coupling (FIG. 6a). We tracked the motion of the particles and the patterns showed that the motion was confined within ±20 nm in xy and 10 nm in z, consistent with the size of the antibody-antigen-antibody complex (about 20 nm) (FIG. 6b). Therefore, we infer the motion is due to the specific binding of TnT. The patterns here share some similarities to those of the DNA tethered particles in FIG. 4, but the tether numbers cannot be estimated from the patterns, because the model is only valid to rigid tethers. The structure of the antibody contains hinges connecting the Fab and Fc domains, which offer flexibility to the structure.

To confirm the particle motion was due to specific binding rather than non-specific adsorption, we performed a control experiment without TnT. The gold surface was functionalized with capture antibody and blocked with BSA, followed by incubation with detection antibody and then the particles. The antibody-antigen-antibody tether could not be formed in the absence of TnT, thus any particles attached to the surface should be attributed to non-specific interactions (FIG. 6c). We tracked the motion of these particles and the results are shown in FIG. 6d. The patterns are notably smaller than those in the specific interaction, indicating the presence of strong restriction from multiple binding sites, which is a feature of non-specific binding.

The capability of distinguishing specific and nonspecific binding based on tether flexibility offers additional benefits in detecting biomarkers in complex media such as serum, which is known to generate dramatic nonspecific interactions. To demonstrate this capability, we measured different concentrations of TnT from 0.268 ng/L to 4200 ng/L in serum and compared the results with conventional digital ELISA, which only counts the number of bound particles. In conventional digital ELISA, we found that the particle counts saturated at high TnT concentration (FIG. 6e), due to the depletion of binding sites for both specific and nonspecific bindings. In principle, specific binding and nonspecific binding compete with each other, thus it is expected that the proportion of specific binding should increase with TnT concentration. In other words, if the nonspecific binding can be removed, the specific binding should reveal a dose-dependent behavior. To verify our hypothesis, we analyzed the motion of each particle using the same set of data and calculated the distance travelled by each particle (or excursion) in xy plane within 5 s, which was a measure of tether flexibility. We treated particles having excursion distance smaller than 2.00 μm as nonspecific and excluded them from the data (see Methods). The result after removing nonspecific binding is shown in FIG. 6f. We found a linear response above noise level to TnT concentrations from 0.486 to 4200 ng/L. The detection limit of 0.486 ng/L was determined by the mean+2SD (standard deviation) of the blank sample (no TnT, and in pure buffer), which was ˜3 times better compared to conventional digital ELISA. For immunoassays in complex media, blocking is always required to reduce nonspecific interactions. Because our method differentiates binding specificity based on particle motion, it is possible to filter out the nonspecific interactions even without using blockers. Next, we performed the above TnT measurement again directly in serum without applying blocking reagent. We found that the surface-bound particles were around several thousands, independent of TnT concentration. We also counted bound particles in pure buffer without TnT as a control, and the number was a few hundred. These observations confirm that serum without blocking introduced high level of nonspecific background that completely covered the specific signals. However, after filtering out particles with low excursion, we found a dose-dependent response from 81.0 to 4200 ng/L. Although the detection limit is worse than the measurement with blocking, the blocking-free measurement demonstrated our tracking-based approach has high tolerance for nonspecific interaction, which can reduce assay complexity by removing the blocking step when high sensitivity is not required and can improve detection limit for clinical samples with extremely high nonspecific background.

To further investigate the difference in tether flexibility, we applied force to the tethered particles using a laminar flow. The flow was generated in a polydimethylsiloxane (PDMS) microchannel mounted on the gold surface. The magnitude of the applied force was controlled by adjusting the flow rate. Four different forces from 1.0 pN to 4.2 pN were applied to the particles. For the specific interaction, the particles were stretched towards the direction of the flow (FIG. 6g). Also, as the force increased, the tether became more tightly stretched and the fluctuation diminished (FIGS. 6h-i). By contrast, the motion of the non-specifically bound particles was not affected much by the force (FIGS. 6j-l). As we further increased the flow rate, both specific and non-specific bonds were ruptured, but at different flow rates (FIG. 6m). We found that over 90% of the non-specifically bound particles were ruptured at 10 pN, whereas 50% specifically bound particles remained on the surface even at 30 pN (FIG. 6n). This observation reflects that specific binding is stronger than non-specific binding in terms of binding force, even though the non-specific binding has multiple binding sites. Note that the sealing between gold surface and PDMS is not strong enough to withstand the high flow rate, so we performed the experiment on a glass surface using similar surface chemistry (see Methods). Both the tether flexibility and the rupture force confirm that only specifically bound particles exhibit dynamic motions.

### DISCUSSION

The current setup has a temporal resolution of up to 1000 fps, which is limited by the speed of the camera. In a shot noise-limited system, the localization precision is a function of photon number scattered by the particle, which can be improved by increasing the incident light intensity. At 1000 fps, ˜1 nm precision in xy and ˜0.1 nm precision in z can be readily achieved with the 15 mW SLED light source. We anticipate that using a faster camera and a brighter light source can further improve the spatial resolution to sub-nanometer in all three dimensions at ˜100 μs frame rate, which will enable us to investigate protein conformation change and single base pair change in DNA. However, in a force-free system, the localization precision is limited by molecular thermal fluctuations (FIG. 5i), which is a few nanometers depending on the molecular size and flexibility. Stretching the molecule using magnetic or optical tweezers can reduce the fluctuation, but the effect on molecular dynamics should be considered as well. Due to the decay of evanescent field in SPR, the localization precision in z is also dependent on the distance to the surface. The precision will reduce ˜10% when z increases from 0 nm to 20 nm. We note that this z-dependent precision should be considered when the tracking range is large.

SPR tracking shows ˜10% linear deviation in the image plane compared to transmitted tracking (FIG. 3g), which may arise from different imaging principles between the two approaches. The whole particle is evenly illuminated in the transmitted field. In contrast, the illumination in SPR is not uniform due to evanescent field. Therefore, the SPR pattern is dependent on z distance, which leads to inaccurate fitting by the functions. The deviation may also be associated with different fitting methods and parameters. Besides fitting the intensity profile, another way to track SPR pattern is taking advantage of the spatial information, however, its localization precision and accuracy remain to be explored. After all, the 10% linear deviation should have little effect in determining the molecular dynamics. If high accuracy is required in certain measurements, combining SPR (z direction) and transmitted imaging (xy directions) using the dual-channel configuration is an alternative solution.

The evanescent field offers SPR superior sensitivity in z direction, but on the other hand, it also confines the tracking range in z. The z-range is only several hundred nanometers, which is limited by the decay constant (d˜100 nm) of the field. Another limitation of SPR tracking is the requirement of using plasmonic material (gold coated cover glass is used in this work).

We believe our method can contribute to improving the sensitivity and specificity in nanoparticle-based immunoassays, especially those associated with clinical samples that are severely interfered with by a wide range of nonspecific reactions. Although various blocking reagents can be used to reduce nonspecific binding, there is no perfect blocking that can eliminate all the nonspecific interactions, even coupled with sophisticated setup and rounds of optimization. In other words, the detection limit is still hindered by nonspecific binding. We expect our strategy including particle tracking and flow washing can alleviate these problems and improve the detection sensitivity and specificity for biosensors.

### CONCLUSIONS

In conclusion, we have demonstrated 3D particle tracking using SPR with sub-nanometer axial precision and milliseconds time resolution. The axial displacement is directly extracted from the scattered light intensity of the particle, requiring no additional optical components. Using the 3D tracking technique, we have studied the dynamics of short DNA and its interaction with an enzyme, and quantified enzyme induced DNA unwinding rate as a function of DNA length change. We have also shown that the specific binding and non-specific binding of antibody can be differentiated by analyzing the motion dynamics. We anticipate SPR 3D tracking technique will expand the understanding of single-molecule dynamics and contribute to the development of single-molecule biosensors.

Methods

Materials

The gold film for SPR imaging was fabricated by coating cover glass (no. 1, VWR) with 1.5 nm Cr followed by 43 nm Au using an e-beam evaporator (PVD 75, Kurt J. Lesker). The functionalized 48 bp DNA was purchased from Integrated DNA Technologies. Methyl-PEG₄-thiol (MT(PEG)4) was purchased from Thermo Fisher Scientific. 1 μm streptavidin coated polystyrene particles and 150 nm streptavidin coated gold nanoparticles were purchased from Bangs Laboratories and Nanopartz respectively. Gold nanoparticle conjugation kit with 100 nm NHS-activated gold nanoparticles was purchased from Cytodiagnostics. RecBCD enzyme was from New England BioLabs. The troponin T detection kit (Elecsys Troponin T Gen 5 STAT) and troponin T (CalSet Troponin T Gen 5 STAT) were purchased from Roche. Reagent diluent (blocking buffer, Douset) was purchased from Sigma Aldrich. 1× phosphate-buffered saline (PBS) was purchased from Corning. Deionized water with a resistivity of 18.2 MΩ·cm was used in all experiments.

Experimental Setup

The plasmonic imaging system was built on an inverted microscope (Olympus IX-81) with a 60× (NA 1.49) oil immersion objective. The light source was a SLED (SLD260-HP-TOW-PD-670, Superlum) with a wavelength of 670 nm. The plasmonic image of the particles was recorded with a CMOS camera (ORCAFlash 4.0, Hamamatsu) at up to 1000 frames per second. Simultaneous transmitted light imaging was achieved by installing an image splitter (OptoSplit II, Cairn Research) between the microscope and the camera. The light source for the transmitted channel was the stocking halogen of the microscope with a green filter at wavelength of 480-550 nm (IF550, Olympus). The shear flow was generated in a PDMS channel (cross-section: 600 μm×25 μm) using a syringe pump (Fusion 100, Chemyx).

Fabrication of DNA Tethered Particles

The gold surface was cleaned with ethanol and deionized water twice followed by annealing with hydrogen flame to remove contaminates. A PDMS cell was placed on the gold surface for holding solutions. The 16 nm DNA was adapted from reference with a sequence of 5′ HS—(CH₂)₆-TAG TCG TAA GCT GAT ATG GCT GAT TAG TCG GAA GCA TCG AAC GCT GAT (SEQ ID NO: 1), where the thiol group was used to bind the gold surface. The complementary strand was modified with a biotin at the 5′ end for capturing the streptavidin coated particles. To immobilize the 16 nm DNA on the surface, a mixture containing 1 nM thiolated single strand DNA and 10 μM MT(PEG)4 in PBS was introduced to the PDMS well and incubated for 1 hour. Then the gold surface was washed with PBS and incubated in 10 nM complementary DNA for 1 hour to allow hybridization. After hybridization, the surface was washed with PBS again and incubated with streptavidin coated 1 μm PS particles at a concentration of 107 particles/ml for 30 min. Then the surface was slowly washed with PBS to remove untethered particles while not breaking the tethered ones.

RecBCD-DNA Interaction

RecBCD was conjugated to NHS-activated 100 nm AuNPs using a AuNP conjugation kit (Cytodiagnostics). After conjugation, the non-specific sites were blocked with 10% BSA for 10 min. Then the AuNPs were centrifuged at 400 g for 30 min and the supernatant was removed. 100 μL 1× NEBuffer 4 (50 mM potassium acetate, 20 mM Tris-acetate, 10 mM magnesium acetate and 1 mM DTT, pH 7.9) was used to resuspend the AuNPs (particle concentration is ˜7.68×10¹⁰/mL). The RecBCD coated AuNPs were stored at 4° C. The 16 nm DNA substrate has the same sequence as mentioned above. The DNA was immobilized on gold surface using the same protocol and density, except that the complementary strand has no biotin group at the 5′ end. After immobilization, the buffer was switched to 1× NEBuffer 4 to match that of RecBCD. ATP used for initiating the reaction was also dissolved in 1× NEBuffer 4.

Surface Preparation for TnT Detection

The cleaned gold surface was treated with a mixture of 1 nM O-(2-Carboxyethyl)-O′-(2-mercaptoethyl)heptaethylene glycol and 10 μM MT(PEG)₄ in PBS overnight. Then the surface was incubated with 50 mM sulfo-N-hydroxysuccinimide (sulfo-NHS) and 200 mM N-ethyl-N′-(3-(dimethylamino)-propyl) carbodiimide hydrochloride (EDC) for 15 min to activate the carboxyl groups. 1.7 nM TnT capture antibody solution was immediately applied to the activated surface and incubated for 30 min. The remaining activated sites were quenched by 1 M ethanolamine at pH 8.5. The surface was then incubated with 0.1% BSA for 15 min to block non-specific binding sites. For TnT binding experiment, 42 pg/mL TnT was introduced to the capture antibody functionalized surface and kept for 30 min to allow binding. Then 1.7 nM biotinylated TnT detection antibody was applied to the captured TnT and incubated for 30 min to form a sandwiched structure. Finally, the streptavidin coated PS particles were introduced and kept for 30 min to bind the biotin groups. For the non-specific binding experiment, after functionalizing the surface with capture antibody and blocking with BSA, the detection antibody was directly added to the system in the absence of TnT, and then incubated with the PS particles for 1 h. Immobilization of TnT capture antibody on glass surface was achieved by silanizing the surface with 1% 3-glycidyloxypropyl)trimethoxysilane in isopropanol overnight followed by incubating with 1.7 nM capture antibody solution for 1 h. Then the surface was blocked with 0.1% BSA for 15 min.

The dose-dependent measurement was performed in a similar way on a gold surface. After functionalized with capture antibody, the surface was blocked with reagent diluent for 15 min, and 100 μL TnT sample was introduced. The TnT stock solution provided by the kit was in serum with a concentration of 4200 ng/L, and we diluted it with PBS to get concentrations ranged from 0 ng/L (pure PBS buffer) to 4200 ng/L (in serum). After 5 min of incubation, the surface was washed with PBS and incubated in 100 μL biotinylated TnT detection antibody solution (from the kit) for 5 min followed by washing again with PBS. Next, 1 μL 150 nm streptavidin coated gold nanoparticles suspended in 100 μL 10 times diluted PBS was spiked into the sample well and incubated for 5 min. Finally, the surface was washed slowly with the diluted PBS to remove unbound particles in solution. The motion of the particles was tracked at 50 fps for 5 s. After measuring each particle's excursion via tracking, a histogram was generated by plotting particle number vs excursion distance in xy plane. An excursion threshold was set empirically to separate specific binding signal (with excursion greater than the threshold) from nonspecific binding background. The optimal threshold position was determined based on the quality of the specific response curve. Note that the filter also removes a part of specific binding events that have excursion distance smaller than the threshold. A filter with low threshold could not efficiently remove the nonspecific binding, however, high threshold could induce digital counting noise due to insufficient particle counts.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of tracking molecular dynamics, the method comprising:
introducing an incident light toward a second surface of a substrate to induce a plasmonic wave at least proximal to a first surface of the substrate, which first surface comprises a population of particles connected to the first surface via one or more first biomolecules; and,
detecting a change in position of one or more of the particles in the population along at least three dimensions over a duration, which three dimensions comprise two substantially lateral dimensions and an axial dimension, from a change in intensity of the incident light reflected at an interface of the first surface of the substrate, thereby tracking the molecular dynamics.

2. The method of claim 1, comprising detecting changes in position of multiple particles in the population substantially simultaneously.

3. The method of claim 1, comprising detecting changes in position of the particles in the population using a plasmonic imaging technique and/or a microscopic imaging technique.

4. The method of claim 1, further comprising detecting the change in position of the particles in the population along a rotational dimension.

5. The method of claim 1, comprising detecting changes in position of the particles in the population with a precision of 10 nanometers or less.

6. The method of claim 1, comprising detecting changes in position of the particles in the population with a precision of less than one nanometer in the axial dimension.

7. The method of claim 1, comprising detecting changes in position of the particles in the population at least in the axial dimension at a frame rate of about one kilohertz (kHz) or less.

8. The method of claim 1, wherein the duration comprises a time resolution of 100 milliseconds or less.

9. The method of claim 1, further comprising one or more second biomolecules connected to at least some of the particles in the population, wherein the method comprises tracking interactions of the second biomolecules with one or more other biomolecules.

10. The method of claim 1, comprising tracking the molecular dynamics in substantially real-time.

11. The method of claim 1, wherein the first biomolecules are label-free.

12. The method of claim 1, comprising determining an axial position of a given particle using the formula I=I₀e−z/d, where I is the mean image intensity, I₀ is the intensity when the given particle is in contact with the first surface and d is the decay constant of an evanescent field that comprises the given particle.

13. A system for tracking molecular dynamics, comprising:
a substrate having a first surface and a second surface opposite the first surface, wherein the first surface comprises a population of particles connected to the first surface via one or more first biomolecules;
an objective lens or a prism disposed proximal to the second surface of the substrate;
a light source configured to introduce light through the objective lens or the prism to induce a plasmonic wave at least proximal to the first surface of the substrate;
a detector configured to collect light reflected from the substrate; and
a controller that comprises, or is capable of accessing, computer readable media comprising non-transitory computer-executable instructions which, when executed by at least one electronic processor, perform at least:
introducing an incident light toward the second surface of the substrate from the light source to induce the plasmonic wave at least proximal to the first surface of the substrate; and,
detecting a change in position of one or more of the particles in the population along at least three dimensions over a duration, which three dimensions comprise two substantially lateral dimensions and an axial dimension, from a change in intensity of the incident light reflected at an interface of the first surface of the substrate.

14. The system of claim 13, wherein the system comprises a surface plasmon resonance microscopy (SPRM) device.

15. The system of claim 13, wherein the non-transitory computer-executable instructions which, when executed by the electronic processor, further perform at least: detecting the change in position of the particles in the population along a rotational dimension.

16. The system of claim 13, wherein the duration comprises a time resolution of 100 milliseconds or less.

17. The system of claim 13, further comprising one or more second biomolecules connected to at least some of the particles in the population.

18. The system of claim 13, wherein the first biomolecules are label-free.

19. The system of claim 13, wherein the substrate comprises an Au coating.

20. A computer readable media comprising non-transitory computer executable instruction which, when executed by at least electronic processor, perform at least:
introducing an incident light toward a second surface of a substrate from a light source to induce a plasmonic wave at least proximal to a first surface of the substrate, which first surface comprises a population of particles connected to the first surface via one or more first biomolecules; and,
detecting a change in position of one or more of the particles in the population along at least three dimensions over a duration, which three dimensions comprise two substantially lateral dimensions and an axial dimension, from a change in intensity of the incident light reflected at an interface of the first surface of the substrate.
